(19) United States
(12) Patent Application Publication
(10) Pub. No.: US 2020/0069801 A1
(43) Pub. Date: Mar. 5, 2020

(54) Enhanced Selective Cellular Stimulation by Ultrasound

(71) Applicant: The Hong Kong Polytechnic University, Kowloon (HK)

(72) Inventors: Lei SUN, Shatin (HK); Zhihai QIU, Shanghang City (CN); Jinghui GUO, Shanghai (CN)

(73) Assignee: The Hong Kong Polytechnic University, Kowloon (HK)

(21) Appl. No.: 16/115,656

(22) Filed: Aug. 29, 2018

(51) Int. Cl. A61K 41/00; A61N 7/00

(52) U.S. Cl. CPC A61K 41/0028; A61M 37/0092; A61N 2007/0004; A61N 7/00

(57) ABSTRACT

There is provided a method of sensitising a eukaryotic cell to ultrasound stimulation, said method comprising increasing the mechano-sensitivity of a transmembrane ion channel of the cell to ultrasound by introducing a plurality of entire exogenous gas vesicles proximal to the surface of the cell.

### FIELD

The present disclosure relates to non-invasive methods and mechanisms of enhanced selective manipulation of cellular activity in specific spatio-temporal regions using ultrasound.

### BACKGROUND

Cells are the basic building blocks and fundamental structural units of multicellular living organisms. In a multicellular complex lifeform, specialized cells perform various functions that are essential to the well-being of the organism, including for example, transportation of nutrients and wastes, generation of energy, defence against invading species and reproduction.

Activation and/or stimulation of cell function has been a topic of interest amongst biologists. In particular, there have been numerous reports on the stimulation of immune cells in attempt to boost defence against infectious diseases in many organisms, including humans. The stimulation of neurons has also received widespread attention, particularly in view of the possibility of improving cognition and memory.

Diverse modalities have been developed in the past few decades in an attempt to stimulate the neural circuits of the human brain, ranging from highly invasive deep brain stimulation (DBS), to less invasive transcranial direct current stimulation (tDCS), transcranial magnetic stimulation (TMS), chemogenetics, optogenetics and ultrasound-based brain stimulation.

Among these techniques of stimulation, ultrasound-based brain stimulation is one promising candidate. Previous research has demonstrated the use of ultrasound to activate mechanosensitive ion channels such as Piezo 1, thereby opening up the ion channel and leading to influx of calcium ion into the cell. As would be appreciated, a wide variety of cell signaling processes depend on intracellular calcium concentration and an increase of calcium ion in the cytoplasm will result in various responses depending on the cell type. For instance, calcium signaling through ion channels is important in neuronal synaptic transmission and may improve learning and memory through altering synaptic plasticity.

However, the present methods of ultrasound-based brain stimulation still have significant challenges. In particular, the focal spot of the ultrasound acoustic wave is too large for single neuron or neuron type stimulation. Without being able to pinpoint specific targeted neurons for stimulation, systematic study on each part and sub-region of the brain is therefore difficult.

There is a need therefore to provide a non-invasive method for stimulation of selected neural circuits for understanding brain function and treating brain disorders with high spatial and temporal precision.

### SUMMARY

Features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims.

An aspect of the present disclosure provides a method of sensitising a eukaryotic cell to ultrasound stimulation. The method comprises increasing the mechano-sensitivity of a transmembrane ion channel of the cell to ultrasound by introducing a plurality of entire exogenous gas vesicles proximal to the surface of the cell.

Advantageously, the method may further comprise modifying the plurality of gas vesicles for localisation proximal to the transmembrane ion channel of the cell prior to introducing the gas vesicles.

Optionally, the modification of the plurality of gas vesicles for localisation may comprise attaching modification peptide having at least one binding domain engageable with a component of the cellular membrane to the gas vesicles.

The modification peptide may be attached to the gas vesicles via an amine group of a gas vesicle protein and may be selected from the group comprising of cell adhesion peptide, an antibody and α-bungarotoxin.

In one embodiment, the cell adhesion peptide may be arginylglycylaspartic acid.

In another embodiment, the antibody may specifically bind to the transmembrane ion channel, which may be selected from the group comprising Piezo 1, Piezo 2, MscL-G22s and CFTR.

Optionally, the exogenous gas vesicles may be derived from prokaryote, phototropic bacteria, non-phototropic bacteria or archaea. In particular, the prokaryote may be a cyanobacteria, for example, Anabaena flos-aquae.

Advantageously, the entire exogenous gas vesicles may be introduced at an amount to give a final concentration of 0.4 nM to 1 nM of the gas vesicles at an extracellular matrix of the cell.

The cellular activity may be stimulated in a time resolved manner in response to ultrasound applied to the cell and the time resolution of the cellular activity stimulation may be in the sub-second region.

Advantageously, application of ultrasound to the gas vesicles proximal to the transmembrane ion channel(s) of the cell may activate said channels to permit calcium passage therethrough causing increased cytoplasmic calcium concentration.

The increased cytoplasmic calcium concentration may result from a simultaneous influx of calcium from the extracellular matrix and release of calcium from intracellular storage of the cell.

The increased cytoplasmic calcium concentration may be detected by fluorescent imaging of cytoplasmic calcium ions.

In one embodiment, the cell may contain calcium sensitive proteins that change configuration according to the environmental calcium concentration.

The transmembrane ion channel may be endogenous to the cell.

Alternatively, additional exogenous mechanosensitive transmembrane ion channels may be expressed by the cell in addition to endogenous transmembrane ion channels.

The gas vesicles may be located at an extracellular space proximal to the surface of the cell.

Optionally, the cell may be a neuron or an immune cell.

In another aspect of the disclosure there is provided a gas vesicle for localisation proximal to a transmembrane ion channel of a cell. The gas vesicle may comprise a modification peptide having at least one binding domain engageable with a component of the cellular membrane.

The modification peptide may be attached to the gas vesicles via an amine group of a gas vesicle protein.

The modification peptide may be selected from the group comprising cell adhesion peptide, an antibody and α-bungarotoxin.

In one embodiment, the cell adhesion peptide may be arginylglycylaspartic acid.

In another embodiment, the antibody may specifically bind to the transmembrane ion channel, which may be selected from the group comprising Piezo 1, Piezo 2, MscL-G22s and CFTR.

Optionally, the gas vesicles may be derived from prokaryote, phototropic bacteria, non-phototropic bacteria or archaea.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In order to describe the manner in which the above-recited and other advantages and features of the disclosure can be obtained, a more particular description of the principles briefly described above will be rendered by reference to specific embodiments thereof which are illustrated in the appended Figures. Understanding that these Figures depict only exemplary embodiments and are not therefore to be considered to be limiting of its scope, the principles herein are described and explained with additional specificity and detail.

Preferred embodiments of the present disclosure will be explained in further detail below by way of examples and with reference to the accompanying Figures, in which:

FIG. 1 depicts a schematic representation of gas vesicles (GV) mediated ultrasound stimulation of mechanosensitive ion channels.

FIG. 2A shows the calcium imaging of neuron cell line CLU199 before GV mediated ultrasound stimulation.

FIG. 2B shows the calcium imaging of neuron cell line CLU199 after GV mediated ultrasound stimulation.

FIG. 2C plots the changes in intracellular calcium concentration observed in FIGS. 2A and 2B before and after GV mediated ultrasound stimulation.

FIG. 2D depicts the GV dosage dependence of increase in intracellular calcium concentration upon ultrasound stimulation.

FIG. 3A schematically depicts amino groups on the surface of GV that are available for further functionalisation by various moieties.

FIG. 3B schematically depicts the binding of an antibody-functionalised GV to a cell surface ion channel.

FIG. 4A depicts the targeting of GVs functionalised with RGD peptides (RGD-GV) to cancer cells MCF-7 overexpressing integrin.

FIG. 4B depicts the calcium fluorescence response of the cells of FIG. 4A in the presence and absence of RGD-GVs.

FIG. 5 depicts an exemplary embodiment for connecting a GV to a Piezo 2 antibody via a linker.

FIG. 6A shows the calcium imaging of primary cultured rat cortical neurons in the presence of Piezo 2 antibody functionalised GVs (Piezo2AB-GVs) before ultrasound stimulation.

FIG. 6B shows the calcium imaging of primary cultured rat cortical neurons in the presence of Piezo 2 antibody functionalised GVs (Piezo2AB-GVs) after ultrasound stimulation.

FIG. 7A depicts respectively the fluorescence image of Piezo2AB-GVs.

FIG. 7B depicts the fluorescence image of neuron cell line CLU199 in the present of Piezo2AB-GVs seen in FIG. 7A upon ultrasound stimulation.

FIG. 7C depicts the calcium fluorescence response of CLU199 neurons in PBS and in the presence of GVs or the Piezo 2 antibody attached GV of FIG. 9A.

FIG. 7D shows the time-resolved calcium fluorescence response of the neurons of FIG. 7B in the presence and absence of Piezo2AB-GVs.

FIG. 8 depicts a schematic representation of a protein domain targeting strategy.

FIG. 9A depicts the fluorescence image of Chinese hamster ovary cells transfected with Piezo 1 gene encoded with bungarotoxin-binding sites (BBS) sequence.

FIG. 9B depicts the fluorescence image of GV functionalised by bungarotoxin (bungarotoxin-GV) targeting to the BBS transfected Chinese hamster ovary cells of FIG. 9A.

FIG. 10 depicts the effect of various concentration of GVs on the change in intracellular calcium concentration upon ultrasound stimulation at predetermined intervals.

FIG. 11A depicts the temporal resolution of intracellular calcium concentration changes upon GV mediated ultrasound stimulation at 20 seconds intervals.

FIG. 11B depicts the temporal resolution of intracellular calcium concentration changes upon GV mediated ultrasound stimulation at 3 seconds intervals.

FIG. 11C shows a summary of the time-resolved calcium fluorescence response to various ultrasound stimulation intervals from 60 s to 100 ms.

FIG. 11D shows a plot of on-set interval against stimulation interval.

FIG. 12A to 12D depict the changes in intracellular calcium concentration upon GV mediated ultrasound stimulation. FIG. 12A shows the calcium fluorescence change in the presence of calcium ion. FIG. 12B shows the calcium fluorescence change in the absence of calcium ion. FIG. 12C shows the calcium fluorescence change in the presence of calcium ion and Ruthenium Red, a calcium signaling blocker. FIG. 12D shows the calcium fluorescence change in the absence of calcium ion and the presence of Ruthenium Red.

FIG. 13 depicts the change in intracellular calcium concentration upon GV mediated ultrasound stimulation in the presence of calcium ion, thapsigargin or Ruthenium Red.

FIG. 14 depicts an exemplary imaging system for observing changes in intracellular calcium concentration.

### DETAILED DESCRIPTION OF PREFERRED EMBODIMENTS

Definitions

“Mechanosensitive” ion channels, are a component of the cellular force-sensation machinery whose opening is controlled by diverse mechanical stimuli such as touch, hearing, crowding, stretch and cell volume, which effectively “translates” physical force into chemical messages.

The piezo-type mechanosensitive ion channel component 1 (Piezo 1) and piezo-type mechanosensitive ion channel component 2 (Piezo 2) are cation channels and are reported to exhibit a preference for calcium ions (Ca2+), whose intracellular levels have been widely used as an indicator for neural activation. The estimated force required to activate Piezo1 can be as low as 10 pN.

Similarly, MscL is a bacterial originated mechanosensitive ion channel, named: The Large Conductance Mechanosensitive Ion Channel (MscL) Family.

As used herein the terms Piezo 1, Piezo 2 and MscL-G22s are taken to mean the full proteins or fragments thereof.

“Gas vesicles” (abbreviated as GVs), or also referred to as “nano-gas vesicles” (abbreviated as NGVs), are hollow structures made of protein and filled with air. Gas vesicles are found in various planktonic microorganisms, in which the GVs provide buoyancy and enable the cell to migrate vertically in water. The shape of GVs is usually a cylindrical tube closed by conical end caps. Two proteins have been shown to be present in the gas vesicles—GvpA makes the ribs that form the structure, and GvpC binds to the outside of the ribs and stiffens the structure against collapse.

It should be understood that a variety of naturally occurring gas vesicles can be employed in the present invention. For example, they can be obtained from various procaryotes, including cyanobacteria such as Microcystis aeruginosa, Aphanizomenon flos aquae and Oscillatoria agardhii; phototropic bacteria such as Amoebobacter, Thiodictyon, Pelodictyon, and Ancalochloris; nonphototropic bacteria, such as Microcyclus aquaticus; and archaea, such as Haloferax mediterranei, Methanosarcina barkeri, Halobacteria salinarium. Preferred procaryotes are filamentous, for ease of separation, and have high vesicle content, by volume, for greater gas delivery or removal capability. Therefore, Anabaena flos aquae, where the vesicles comprise about 10% of the volume of the cell, is a preferred source of naturally occurring gas vesicles.

“Transgenic methods” is meant a method of introduction of recombinant nucleic acid molecules into the genome of the organism.

“Defined spatial region” is meant a predetermined specific and confined area in the brain.

A preferred embodiment for genetically targeted expression of the mechanosensitive proteins of the present disclosure comprises a vector which contains the gene for such protein.

As used herein, the term “vector” refers to a nucleic acid molecule capable of transporting another nucleic acid to which it has been operatively linked between different genetic environments.

The term “vector” also refers to a virus or organism capable of transporting nucleic acid molecules. Preferred vectors are can replicate/express the nucleic acids to which they are linked. Vectors capable of directing the expression of genes to which they are operatively linked are referred to herein as “expression vectors”. Other preferred vectors include viruses such as recombinant AAV virus. Preferred vectors can genetically insert mechanosensitive proteins into both dividing and non-dividing cells; in-vivo or in-vitro.

Those vectors can include a prokaryotic promoter capable of directing the expression (transcription and translation) of the protein in a bacterial host cell, such as E. coli. A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment according to an aspect of the present disclosure.

Expression vectors compatible with eukaryotic cells, can also be used. Eukaryotic cell expression vectors are well known and available from several commercial sources. Typically, such vectors are provided containing convenient restriction sites for insertion of the desired DNA homologue.

One preferred embodiment of an expression vector of the present disclosure is a rAAV virus comprising the gene for the mechanosensitive ion channel together with a hSyn promoter. This vector is used in one aspect of the present disclosure to deliver genes to specific neurons.

“Protein” in this sense includes proteins, polypeptides, and peptides. Also included within the protein of the present disclosure are amino acid variants of the naturally occurring sequences, as determined herein. Preferably, the variants are greater than about 75% homologous to the protein sequence of Piezo1, MscL, and more preferably greater than about 80%, even more preferably greater than about 85% and most preferably greater than 90%. In some embodiments the homology will be higher.

Various embodiments of the disclosure are discussed in detail below. While specific implementations are discussed, it should be understood that this is done for illustration purposes only. A person skilled in the relevant art will recognize that other components and configurations may be used without departing from the scope of the disclosure.

The present disclosure provides an improved method of stimulating cell activity and signaling by using ultrasound in combination with GVs. The method of gas vesicle mediated ultrasound stimulation of cells as described herein addresses the need in the art for non-invasive, precisely controllable, wide field of view stimulation of cells, for example neurons, in order to boost cell function or to study the mechanistic pathway of cell function.

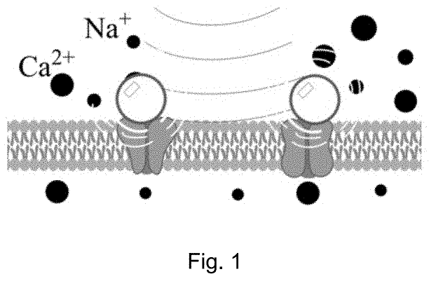

FIG. 1 illustrates schematically the strategy of using GVs for sensitising a cell's response to ultrasound stimulation. As shown in the figure, a number of trans-membrane mechanosensitive ion channels are present on the cell surface. These ion channels may be endogenous or exogenous. Although the underlying mechanism of GV-mediated ultrasound stimulation is not entirely clear, it is believed that the presence of GVs in the proximity of the cell surface ion channels alters the influence of the ultrasound or amplify the sound wave near the mechanosensitive ion channels. As a result of the mediation by the GVs, the sensitivity of the ion channels to the ultrasound stimulus is enhanced.

In one embodiment, the GVs may be simply dispensed in the extracellular matrix without any additional modifications. In yet another embodiment, the GVs may be functionalised with a moiety that targets the GVs to the cell, or to a mechanosensitive ion channel on the cell membrane. Due to the exogenous nature of the GVs, the GVs remain at the extracellular space proximal to the cell, and do not enter the cells.

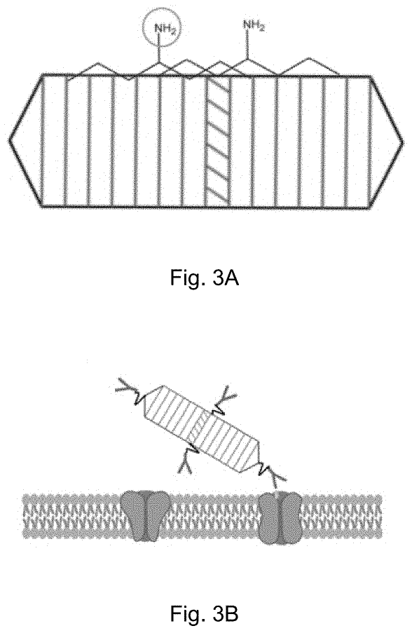

FIG. 3A is a schematic illustration for a GV produced by Anabaena flos-aquae. The GV comprise two proteins, namely GvpA and GvpC. Free amine ends of the GV proteins are also shown in the figure.

The GV may be functionalised with various protein modification strategies known in the art. For instance, a chemical or a peptide may be connected to the GV via the free amine ends on the GV protein. The chemical or peptide may be recognized by a cell surface receptor and binds specifically to the receptor. Alternatively, the chemical or peptide may itself be a receptor and specifically recognises a cell surface protein.

In a preferred embodiment, the peptide that is linked to the GV may be a cell adhesion peptide, including but not limited to Arginylglycylaspartic acid (RGD). The cell adhesion peptide is recognised by a receptor on the cell surface, e.g. integrins, thus bringing the GV to the proximity of the cell membrane.

In another preferred embodiment, the GV is linked to an antibody that targets a cell surface protein. For example, the antibody may be a Piezo 2 antibody that targets the cell surface Piezo 2 mechanosensitive ion channels.

FIG. 3B depicts the strategy for functionalisation of the GV with an antibody by attachment via the free amine ends depicted in FIG. 3A. However, a person skilled in the art would appreciate that the antibodies may be attached to the GV by any conventional method known in the art, and the attachment via the free amine end should be understood as a non-limiting example.

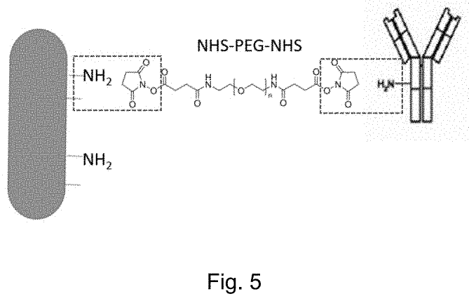

FIG. 5 is a schematic illustration of the attachment of a Piezo2 antibody to a free amino end of a GV via a NHS-PEG-NHS linker. Although the compound NHS-PEG-NHS is illustrated as the linker connecting the GV and the antibody, a person skilled in the art would appreciate that any compounds that may form bonding with the GV and Piezo2 antibody may be used to connect the antibody to the GV.

In addition to Piezo 2, the GV may be functionalised with various antibodies that target a cell membrane protein. For example, the antibody may target an ion channels other than Piezo 2 on the cell membrane. In a preferred embodiment, the ion channel may be a mechanosensitive ion channels, including but not limited to Piezo1, Piezo2, Large Conductance Mechanosensitive Ion Channel (MscL) and Cystic Fibrosis Transmembrane Conductance Regulator (CFTR).

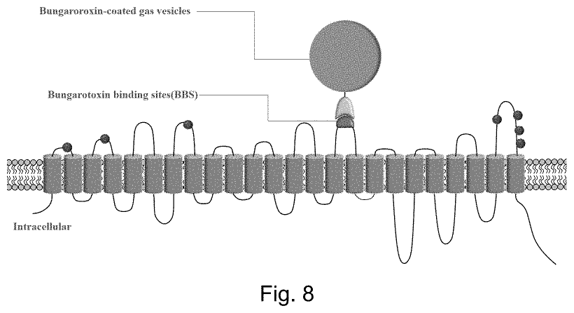

In yet another preferred embodiment schematically illustrated in FIG. 8, a domain level targeting strategy utilises BBS sequence transfection and GV attached with bungarotoxin. In this example, cells may be transfected with bungarotoxin-binding sites (BBS) sequences at the Piezo 1 gene. The GVs are connected to bungarotoxin, which recognise the transmembrane BBS co-expressed with Piezo 1 by the transfected cell. As a result, the GV is targeted to a specific domain on the Piezo 1 (where the BBS protein is located) at the cell surface.

The presence of GVs in the proximity of cell surface, and in particular in the proximity of a mechanosensitive ion channel (regardless of whether assisted by the binding between a peptide linked to the GV and a cell surface protein), enhances the ion channel's response to ultrasound stimulation. While the underlying mechanism is not entirely clear, it is postulated that GVs enhance the vibrations from the ultrasound source, thus transmitting a higher level of stimuli to the mechanosensitive ion channel and/or the cell.

Depending on the type of the cell and the mechanosensitive ion channel being stimulated, various response may be obtained upon ultrasound application. For example, in the presence of GV, ultrasound stimulation of the Piezo ion channel (e.g. Piezo 1 and Piezo 2) result in opening of the ion channel allowing an influx of calcium into the cell from the extracellular matrix. As would be appreciated, increase in intracellular calcium concentration may activate a number of pathways leading to alteration in a number of cell functions.

The methods of controlling cell properties of the present disclosure will result in the ability to probe function in intact neural circuits, allowing for the understanding of the role of particular neurons in animal models including of learning, memory and motor coordination. This will enable the discovery of drugs capable of modulating whole-circuit function, essential for the addressing of complex neurological and psychiatric diseases. For the first time, genetically-targeted neurons within animals will be addressable by ultrasound allowing examination behavioral or circuit-dynamics function and allow for observed normal and dysfunctional behaviours.

In some embodiments it would be appreciated that the specific spatial and temporal control of neurons that is provided by the present disclosure could potentially provide the ability to target neural circuits non-invasively for understanding brain functions and dysfunctions, as well as providing a treatment strategy, which can hopefully translate to clinical practice.

Control over synaptic events through regulation of mechanosensitive channels expressed endogenously or introduced therein enables the potential for control over cell plasticity, learning and memory. Potentially similar such mechanisms can be utilised to explore and/or treat neurological disorders such as Parkinsons disease, Alzheimers disease, diabetes, neuro-immuno modulation.

### EXAMPLES

1. Cell Culture

Embryonic mouse hippocampal cell line mHippoE-18 (referred to in the text as “CLU199”) was purchased from Cellution biosystem, Cedarlane Laboratories. CLU199 cells were maintained in Dulbecco's Modified Eagle Medium (DMEM) (high glucose and no sodium pyruvate), supplemented with 10% fetal bovine serum, 100 units/mL penicillin and 100 μg/mL streptomycin (all from Gibco), inside a humidified incubator 37° C. with 5% CO2.

2. Primary Cortical Neuron Harvest

Primary cortical neurons from embryonic mice brains were harvested as described previously with slight modifications. Briefly, pregnant mice were sacrificed at E16.5-E17, and brains from the embryonic mice were collected. They were then dissected under a microscope to separate the cortex from the other brain mass. Cortical cells were dispersed and seeded into culture dishes or collagen-coated glass

3. Preparation of GVs

Anabaena flos-aquae was cultured in sterile BG-11 medium at 25° C. under fluorescence lighting with 14 hours/10 hours light/dark cycle. GVs were isolated by hypertonic lysis to release GVs with quickly adding sucrose solution to a final concentration of 25%. GVs were isolated by centrifugation at 400×g for 3 hours after hypertonic lysis. To purify the GVs solution, it was washed by the same centrifugation process 3 times and stored in PBS at 4° C. GVs solution concentration was measured by optical density at 500 nm (OD500) by UV-Visible spectrophotometer.

4. Calcium Imaging of CLU199 Neuron Cell Line Under GV Mediated Ultrasound Stimulation

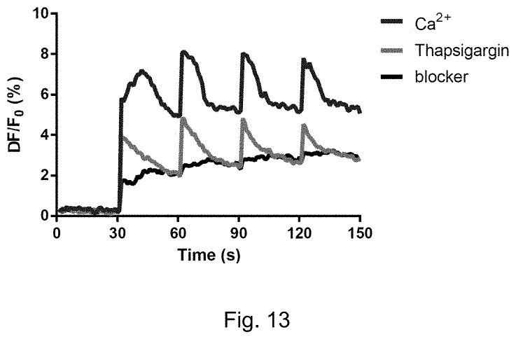

GVs were added to CLU199 neuron cell line and the neurons were incubated with Fluo-4 AM from Thermo Fisher at 37° C. for 30 minutes. Ultrasound at 1 MHz frequency and 0.5 MPa intensity (300 ms chirped at 1 Khz with 0.5% duty circle) was then applied and the fluorescent images recorded using the calcium imaging system of FIG. 13.

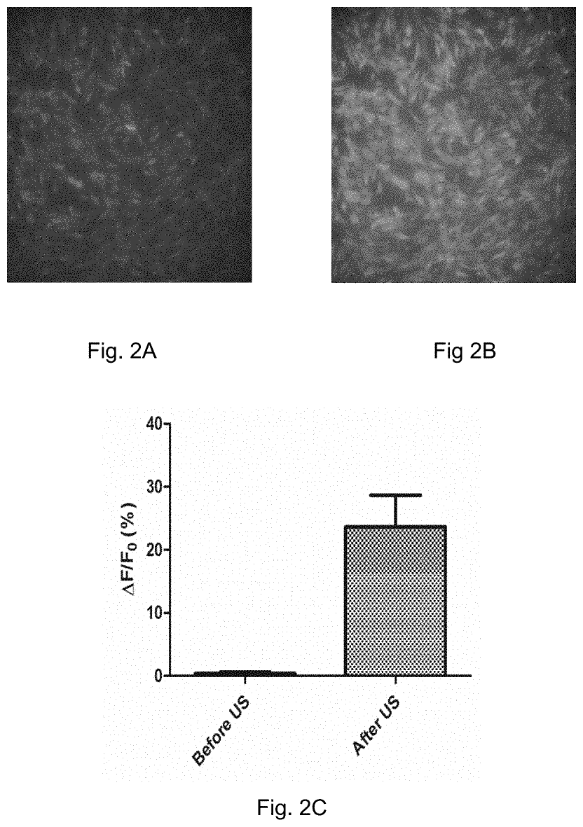

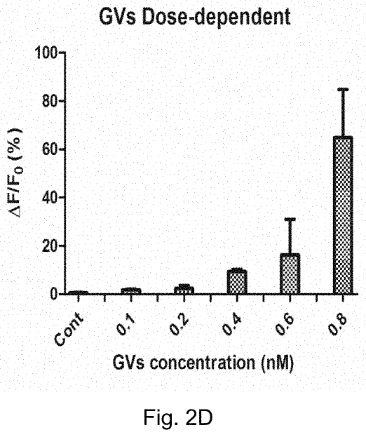

FIGS. 2A and 2B show the calcium imaging obtained before and after ultrasound stimulation (with 0.4 nM GVs). The enhancement of the fluorescence intensity in the cell body of FIG. 2B signifies the influx of calcium ion, which is a result of the opening of mechanosensitive ion channels such as Piezo 1, Piezo2 and G-protein-coupled receptors (GPCR) on the cell surface due to GV medicated ultrasound stimulation.

FIG. 2C plots the changes in intracellular calcium concentration (measured by changes in the fluorescence intensity) before and after the application of ultrasound stimulation.

FIG. 2D depicts the changes in intracellular calcium concentration (measured by changes in the fluorescence intensity) mediated by various concentration of GVs upon ultrasound stimulation. It can be seen that the intracellular calcium concentration increases with higher concentration of GVs, thus demonstrating that the opening of mechanosensitive ion channel and influx of calcium is mediated by GVs. In particular, at a GV concentration of 0.4 nM or above, significant increase in calcium fluorescence could be observed.

5. Functionalisation of GVs

A. Functionalisation with RGD Peptide/Folic Acid—Cell Level Targeting

GVs were functionalised by RGD peptide. For the RGD functionalised GV (RGD-GV) synthesis, firstly, SMCC (1 mM) was added to pure GV solution (molar ratio: SMCC/GV=2000/1) in PBS (pH=7.2). After 1 h shaking reaction in room temperature, and then, the intermediate nanoparticles were purified by centrifuge for 4 times by PBS buffer. RGD (1 mM) was added to SMCC-GV solution (molar ratio: RGD/GV=1000/1) in PBS. Reacting in room temperature for 2 hours with shaking and then put in 4° C. for 20 hours. After that, the RGD-GV nanoparticles were purified by centrifuge for 5 times by PBS buffer. The resultant nanoparticles were stored in PBS buffer. Protoporphyrin IX (PPIX or PpIX) was then tagged to the GV. Firstly, EDC (10 mM) and sulfo-NHS (25 mM) were added to PpIX solution (1 mM) in 0.1 M sodium phosphate (pH=7.4). After 15 mins reaction in room temperature, the solution together with NHS-PEG-NHS was added to pure GV solution (molar ratio: PpIX/PEG/GV=500/500/1) and followed by mixture for 2 hours in room temperature. Then, the intermediate nanoparticles were purified by centrifuge for 3 times by PBS buffer. The resultant nanoparticles were stored in PBS buffer.

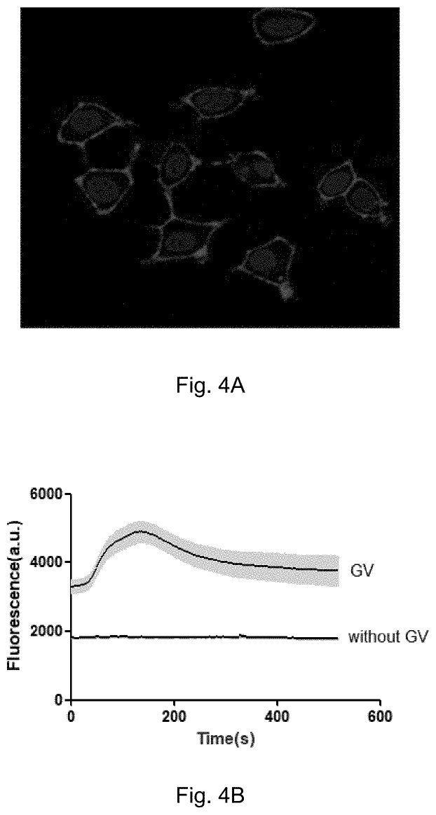

FIG. 4A shows the cell level targeting of RGD functionalised GVs (RGD-GVs) tagged with PPIX. The RGD-GVs were incubated with MCF-7 cells (which overexpress integrin) for 2 hours. And then stained with propidium iodide (PI) to visualize the nuclei followed by confocal microscopy. The fluorescence around the nuclei indicates the position of the GVs, which surrounds the cells as a result of the adhesion of the GV to the cell via the binding between RGD and integrin.

FIG. 4B shows the calcium fluorescence response of the cells of FIG. 4A in the presence and absence of RGD-GVs. An increase in the calcium fluorescence response is only seen in the presence of RGD-GVs.

In another example, folic acid (FA) and PPIX were immobilized to GVs' protein shell by covalent conjugate. For the FA-PPIX-GV synthesis, firstly, EDC (10 mM) and sulfo-NHS (25 mM) were added to PPIX solution (1 mM) in 0.1 M sodium phosphate (pH=7.4). After 15 mins reaction in room temperature, the solution together with NHS-PEG-NHS was added to pure GV solution (molar ratio: PpIX/PEG/GV=500/500/1) and followed by mixture for 2 hours in room temperature. Then, the intermediate nanoparticles were purified by centrifuge for 3 times by PBS buffer and mixed with folic acid (molar ratio: FA/particle=500/1). The mixture was shaken for another 2 hours and followed by purification by centrifuge for 4 times. The resultant nanoparticles were stored in PBS buffer.

In vitro cellular attachment experiments were performed using MCF-7 cells, known to express the folate receptor. To investigate the efficacy of folic acid coating on the attachment of GV based sonosensitizer, folic receptor positive MCF-7 cells were incubated with FA-PpIX-GV, PpIX-GV (i.e. not attached with FA) and pure PBS, with PpIX-GV and PBS serving as control groups. The final results were acquired by using confocal microscopy. From the results, red fluorescence intensity which came from PpIX was significantly increased in FA-PpIX-GV group compared to non-targeting PpIX-GV group. After 1 hr incubation, most of the sensitizers (PpIX-GV and FA-PpIX-GV) were located on cell membrane instead of cytoplasm.

B. Functionalisation with Piezo2 Antibody—Protein Level Targeting

Synthesis of Piezo2 Antibody Functionalised GVs (Piezo2AB-GVs)

EDC (10 mM) and sulfo-NHS (25 mM) were added to Piezo2 antibody solution (1 mM) from Thermo Fisher in 0.1 M sodium phosphate (pH=7.4). After 15 mins reaction in room temperature, the solution together with NHS-PEG-NHS was added to pure GV solution (molar ratio: antibody/PEG/GV=500/500/1) and followed by mixture for 2 hours in room temperature. Then, the intermediate nanoparticles were purified by centrifuge for 3 times by PBS buffer and mixed with folic acid (molar ratio: antibody/particle=500/1). The mixture was shaken for another 2 hours and followed by purification by centrifuge for 4 times. The resultant nanoparticles were stored in PBS buffer.

Calcium Imaging of Primary Cultured Neurons Under Piezo2AB-GVs Mediated Ultrasound Stimulation

Piezo2AB-GVs were added to primary cortical neurons with a final concentration of Piezo2AB-GVs being 0.4 nM and the neurons were incubated with Fluo-4 AM from Thermo Fisher at 37° C. for 30 minutes. Ultrasound at 1 MHz frequency and 0.5 MPa intensity was then applied and the fluorescent images recorded using the calcium imaging system of FIG. 13.

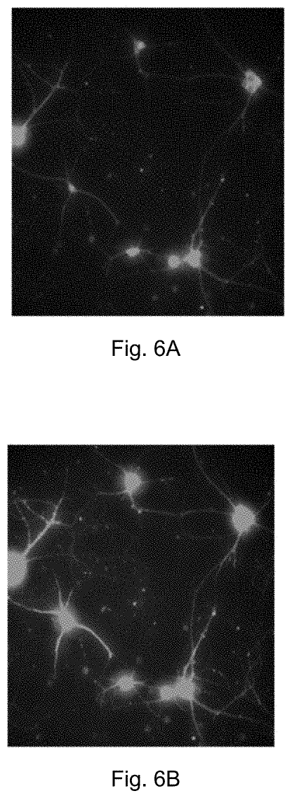

FIGS. 6A and 6B show the calcium imaging before and after ultrasound stimulation. The enhancement of the green fluorescence in the cell body of FIG. 3B signifies the influx of calcium ion, which is a result of the opening of the Piezo2 channel due to Piezo2AB-GV medicated ultrasound stimulation.

Calcium Imaging of CLU199 Under Piezo2AB-GVs Mediated Ultrasound Stimulation

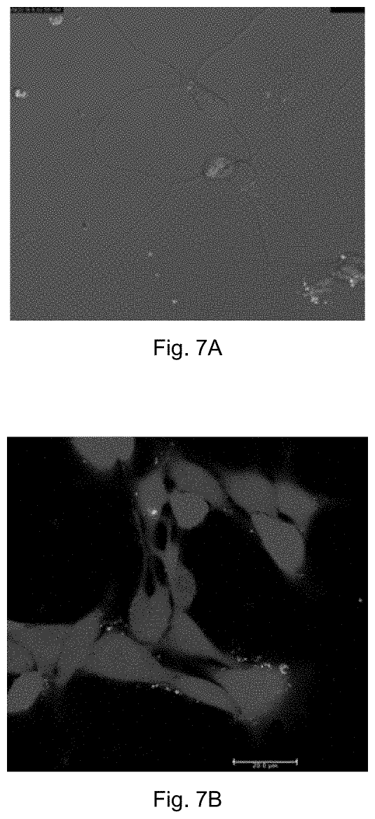

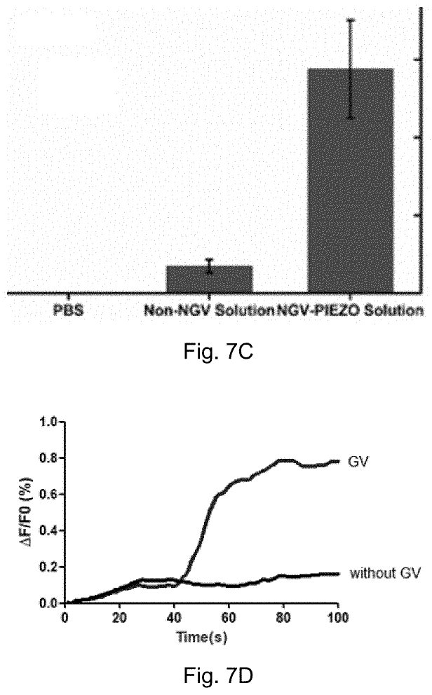

FIG. 7A shows the green fluorescence from Piezo2AB-GVs. In FIG. 7B, CLU199 neurons were incubated with 2.5 mM Cal-590 from AAT Bioquest at 37° C. for 1 hour, and the red fluorescence of FIG. 7B shows the intracellular calcium change upon ultrasound stimulation (1 MHz frequency, 0.5 MPa intensity, 300 ms chirped at 1 Khz with 0.5% duty circle) in the presence of Piezo2AB-GVs, suggesting the neurons were activated by ultrasound and resulting in influx of calcium.

In a further experiment, Piezo2AB-GVs were added to neuron cell line CLU199 and the neurons were incubated with Fluo-4 AM from Thermo Fisher at 37° C. for 30 minutes. Ultrasound at 1 MHz frequency and 0.5 MPa intensity was then applied and the fluorescent images recorded using the calcium imaging system of FIG. 13.

FIG. 7C shows the changes in calcium fluorescence intensity when PBS, GVs (NOT functionalised with Piezo2 antibody) and GVs functionalised with Piezo2 antibody were added to the neurons of FIG. 7B. In PBS (control) or in the presence of GVs (NOT functionalised with Piezo2 antibody), nil or minimal enhancement of calcium fluorescence intensity was observed. In contrast, when the GVs are functionalised with Piezo2 antibody, significant enhancement of fluorescence signal is observed, indicating the opening up of calcium ion channel which is mediated by the GVs. The much higher level of fluorescence signal obtained when the GVs are functionalised with Piezo2 antibody also suggest that the functionalisation assists the GVs to attach itself to the Piezo2 mechanosensitive ion channel on the cell surface.

FIG. 7D shows the time-resolved calcium fluorescence response of the neurons of FIG. 7B in the presence and absence of Piezo2AB-GVs. Significant enhancement of fluorescence response indicating calcium influx is only seen when the neurons are targeted by Piezo2AB-GVs.

C. Functionalisation with α-Bungarotoxin—Domain Level Targeting

Bungarotoxin-binding sites (BBS) sequences were designed to be inserted into the plasmids. Chinese hamster ovary cells from ATCC transfected with Piezo 1 gene encoded with BBS sequence were washed twice with PBS and incubate for 3 h at 37° C. in PBS containing 4 nM α-bungarotoxin conjugated gas vesicles (prepared by adding α-bungarotoxin to GVs and incubating for 2 hours) and 10 mM HEPES. Cells were then washed 3 times with PBS. Calcium fluorescence imaging was obtained with 63× oil lenses of confocal microscope.

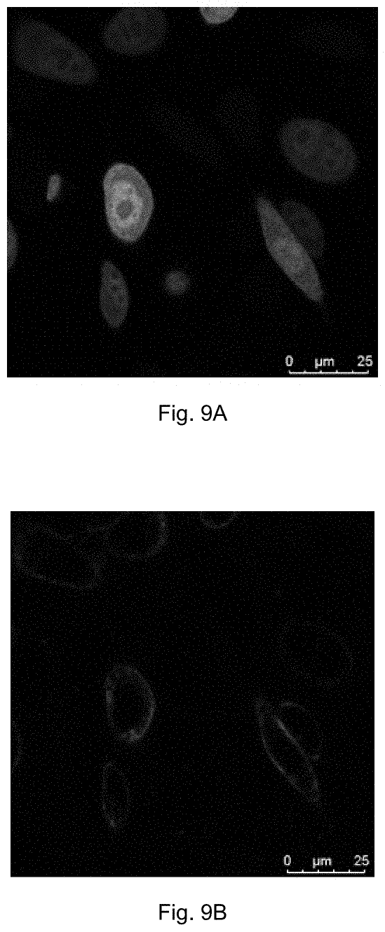

FIG. 9A is the fluorescence image of the cells showing green fluorescence from Piezo 1 (from an enhanced green fluorescent protein encoded into the sequence that serves as an indicator of expression of Piezo1), indicating that the cells were successfully transfected with the BBS sequence at the Piezo 1 gene. FIG. 9B is another fluorescence image of the cells showing red fluorescence from bungarotoxin, which were seen at the peripheral of the BBS transfected cells. This suggests that the α-bungarotoxin and the GVs attached thereto are brought to the close proximity of the cells.

6. Time-Resolved Responses of Calcium Influx

A. Time Resolved Response Studies

GVs were added to CLU199 neurons and the neurons were incubated with Fluo-4 AM from Thermo Fisher at 37° C. for 30 minutes. Ultrasound at 1 MHz frequency and 0.5 MPa intensity (300 ms chirped at 1 Khz with 0.5% duty circle) was then applied at predetermined intervals and the changes in fluorescent intensity recorded using the calcium imaging system of FIG. 13.

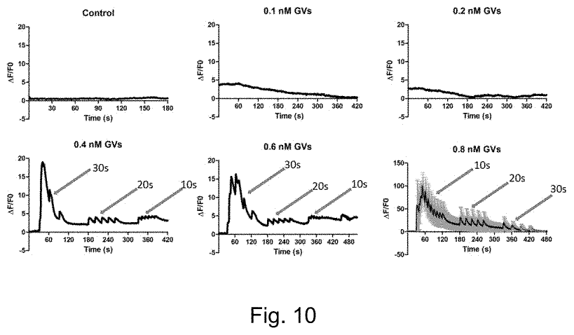

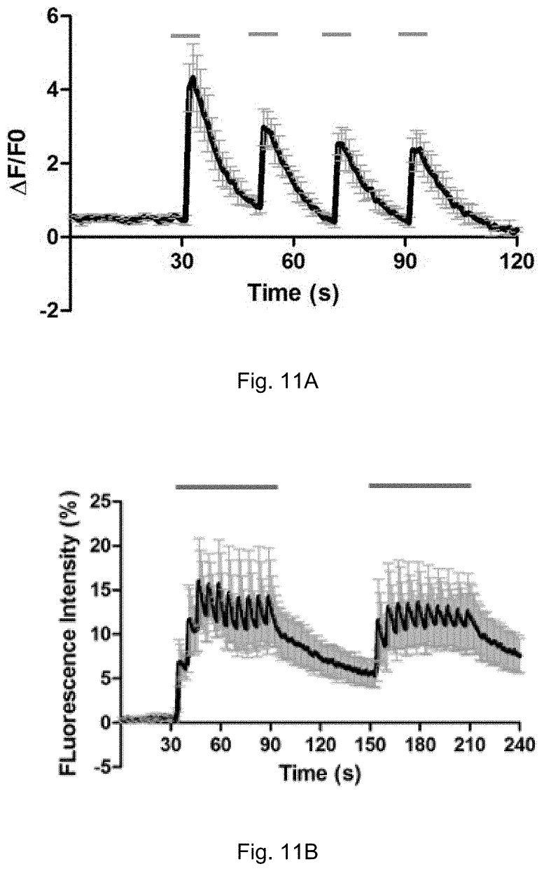

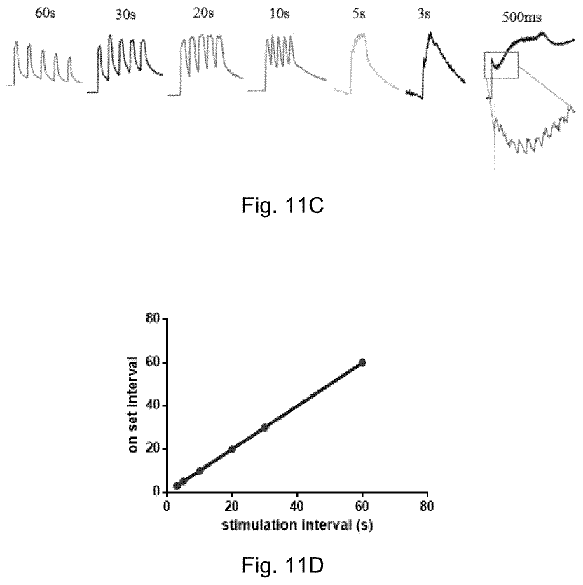

FIGS. 10, 11A and 11B depict the changes in intracellular calcium concentration (measured by changes in the fluorescence intensity) in various concentration of GVs and upon ultrasound stimulation at various time intervals as indicated in the figures.]

FIG. 11C shows a summary of the time-resolved calcium fluorescence response to various ultrasound stimulation intervals from 60 s to 100 ms. As seen from the figure, the calcium responses onset can be as fast as 500 ms. FIG. 11D shows a linear correlation between ultrasound stimulation interval and measured calcium onset interval.

B. Absence of Calcium and/or Presence of Blocker

Time-resolved GV-mediated ultrasound stimulation of CLU199 was conducted using similar procedures as described in Part A of Example 6.

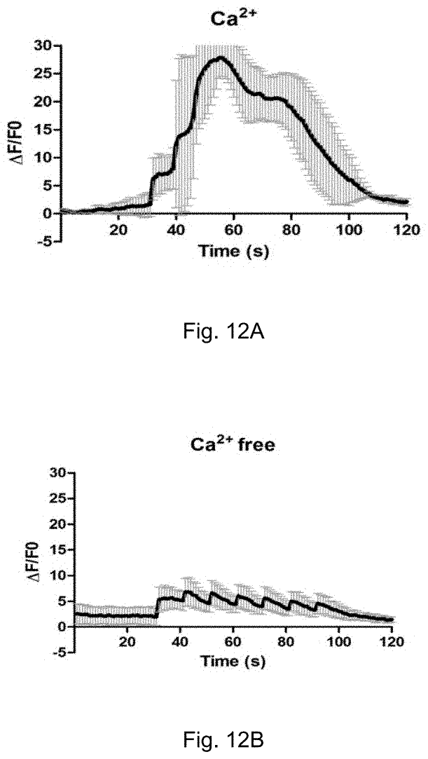

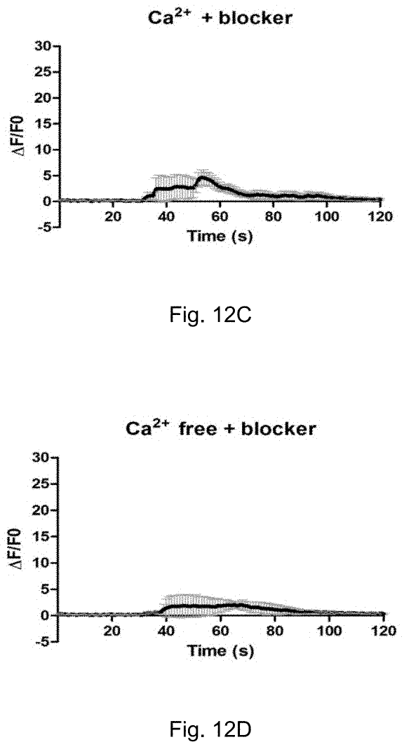

FIG. 12A depicts the changes of intracellular calcium concentration upon GV-mediated ultrasound stimulation. FIG. 12B depicts the changes of intracellular calcium concentration when calcium ion is removed from the extracellular media. Although FIG. 12B shows a significant decrease of the response, influx of calcium is still observable upon ultrasound stimulation which suggests intracellular calcium release. FIG. 12C depicts the response in the presence of calcium and 30 uM Ruthenium Red, a calcium signaling blocker. FIG. 12D shows the results in the absence of calcium and the presence of 30 uM Ruthenium Red.

7. Intracellular Calcium Release

GVs were incubated with CLU199 neuron for 2 hours. The neurons were then treated with either Thapsigargin or Ruthenium Red, followed by ultrasound stimulation.

FIG. 13 shows the changes in calcium fluorescence in Thapsigargin/Ruthenium Red treated neurons upon ultrasound stimulation, and compared to the calcium fluorescence response from CLU199 neurons in artificial cerebral spinal fluid upon GV-mediated ultrasound stimulation. Thapsigargin can release intracellular calcium storage. As a result, upon Thapsigargin treatment the intracellular calcium storage is depleted. Upon subsequent ultrasound application, there is no intracellular calcium storage in the cells, and the observed calcium response represents solely calcium influx from extracellular solution (and not from intracellular storage).

On the other hand, Ruthenium Red blocks the extracellular calcium influx, thus the changes in fluorescence intensity upon ultrasound application in the presence of Ruthenium Red represents the intracellular calcium release stimulated by ultrasound (without any contribution from extracellular calcium influx).

As seen in FIG. 13, the combination of the response from the Ruthenium Red treated cells (amounting to intracellular calcium release stimulated by ultrasound) and the response from the Thapsigargin treated cells (amounting to influx of calcium from extracellular matrix stimulated by ultrasound) is approximately equal to the response from GV-mediated ultrasound stimulation in the absence of Thapsigargin/Ruthenium Red treatment, thus suggesting that the method of present disclosure can mediate both intracellular calcium release and extracellular calcium influx by opening mechanosensitive ion channels on the cell membrane

8. Calcium Imaging System

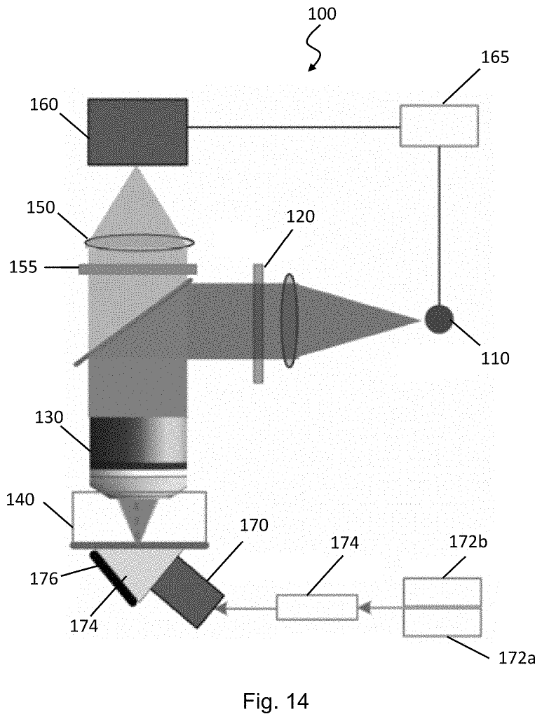

FIG. 14 depicts an exemplary calcium imaging system 100 consisting of a modified upright epifluorescence microscope.

The excitation light was generated by a dual-color LED 110, filtered by 488 nm bandpass filters 120 and delivered via the objective lens 130 to the sample in the sample holder 140 for illuminating the calcium sensor.

The fluorescence signals from the cells were collected by a water immersion objective (UMPlanFLN, Olympus) 130, filtered by a filter wheel with green (525 nm) or red (633 nm) channels 155 LPF; focussed by a tube lens 150 and captured by a sCMOS camera 160 (ORCA-Flash4.0 LT Plus C114400-42U30, Hamamatsu) operated by a controller 165. To minimize phototoxic effects, the LEDs were triggered at 1 Hz and synchronized with sCMOS time-lapse imaging.

The ultrasound stimulation system consisting of a commercial transducer 170 (I7-0012-P-SU, Olympus), two function generators 172a,172b, and a power amplifier 174 (Electronics and Innovation, A075) to produce 200 tone burst pulses at a center frequency of 500 kHz and a repetition frequency of 1 kHz with a duty cycle of 40%. The output intensity was limited to 0.1-0.6 MPa. These parameters are similar to which has been reported to effectively evoke behaviour responses (3). To deliver ultrasound, a triangle waveguide 174 was attached to the ultrasound transducer 170 and placed under the culture dish at a 45-degree angle to the horizontal axis. The other site of the waveguide was mounted with an acoustic absorber 176 to minimize acoustic reverberation. During calcium imaging, the cells were placed in a buffer solution with 130 mM NaCl, 2 mM MgCl2, 4.5 mM KCl, 10 mM Glucose, 20 mM HEPES, and 2 mM CaCl2, pH 7.4.

1. A method of sensitising a eukaryotic cell to ultrasound stimulation, said method comprising increasing the mechano-sensitivity of a transmembrane ion channel of the cell to ultrasound by introducing a plurality of entire exogenous gas vesicles proximal to the surface of the cell.

2. The method of claim 1, further comprising modifying the plurality of gas vesicles for localisation proximal to the transmembrane ion channel of the cell prior to introducing the gas vesicles.

3. The method of claim 2, wherein modifying the plurality of gas vesicles for localisation comprises attaching modification peptide having at least one binding domain engageable with a component of the cellular membrane to the gas vesicles.

4. The method of claim 3, wherein the modification peptide is attached to the gas vesicles via an amine group of a gas vesicle protein.

5. The method of claim 3, wherein the modification peptide is selected from the group consisting of cell adhesion peptide, an antibody and α-bungarotoxin.

6. The method of claim 5, wherein the cell adhesion peptide is arginylglycylaspartic acid.

7. The method of claim 5, wherein the antibody specifically binds to the transmembrane ion channel.

8. The method of claim 6, wherein the transmembrane ion channel is selected from the group consisting of Piezo 1, Piezo 2, MscL-G22s and CFTR.

9. The method of claim 1, wherein the exogenous gas vesicles are derived from prokaryote, phototropic bacteria, non-phototropic bacteria or archaea.

10. The method of claim 1, wherein the entire exogenous gas vesicles are introduced at an amount to give a final concentration of 0.4 nM to 1 nM of the gas vesicles at an extracellular matrix of the cell.

11. The method of claim 1, wherein the cellular activity is stimulated in a time resolved manner in response to ultrasound applied to the cell.

12. The method of claim 11, wherein the time resolution of the cellular activity stimulation is in the sub-second region.

13. The method of claim 1, wherein the cell contains calcium sensitive proteins that change configuration according to the environmental calcium concentration.

14. A gas vesicle for localisation proximal to a transmembrane ion channel of a cell, the gas vesicle comprises a modification peptide having at least one binding domain engageable with a component of the cellular membrane

15. The gas vesicle of claim 14, wherein the modification peptide is attached to the gas vesicles via an amine group of a gas vesicle protein.

16. The gas vesicle of claim 14, wherein the modification peptide is selected from the group consisting of cell adhesion peptide, an antibody and α-bungarotoxin.

17. The gas vesicle of claim 16, wherein the cell adhesion peptide is arginylglycylaspartic acid.

18. The gas vesicle of claim 16, wherein the antibody specifically binds to the transmembrane ion channel.

19. The gas vesicle of claim 18, wherein the transmembrane ion channel is selected from the group consisting of Piezo 1, Piezo 2, MscL-G22s and CFTR.

20. The gas vesicle of claim 14, wherein the gas vesicles are derived from prokaryote, phototropic bacteria, non-phototropic bacteria or archaea.
